(19) United States
(12) Patent Application Publication
(10) Pub. No.: US 2013/0095563 A1
(43) Pub. Date: Apr. 18, 2013

(54) METHOD AND SYSTEM FOR DETECTING AN ANALYTE

(71) Applicants: Timothy J. Imholt, Methuen, MA (US); Susan N. Gottschlich, Marlborough, MA (US)

(72) Inventors: Timothy J. Imholt, Methuen, MA (US); Susan N. Gottschlich, Marlborough, MA (US)

(21) Appl. No.: 13/397,260

(22) Filed: Feb. 15, 2012

Related U.S. Application Data
(60) Provisional application No. 61/547,280, filed on Oct. 14, 2011.

(51) Int. Cl. C12M 1/34; G01N 1/28

(52) U.S. Cl. 435/287.1; 427/2.11

(57) ABSTRACT

Aspects and embodiments are directed to methods and systems of detecting an analyte present in the environment. More particularly, this disclosure relates to methods and systems of detecting a threatening or dangerous analyte that may increase survivability of an individual or group of individuals.

### CROSS-REFERENCE TO RELATED APPLICATION

This application claims priority under 35 U.S.C. §119(e) of U.S. Provisional Patent Application Ser. No. 61/547,280, filed on Oct. 14, 2011, titled “METHOD AND SYSTEM FOR DETECTING AN ANALYTE,” the entire disclosure of which is hereby incorporated herein by reference in its entirety for all purposes.

### BACKGROUND

Many substances in the environment may pose a threat to living beings. These threats, which may include chemical and biological substances or compounds may pose a threat to, for example military personnel or to civilians. A sensing device may be used to detect the threat and to increase survivability of an individual or group.

### SUMMARY

Aspects and embodiments are directed to methods and systems of detecting an analyte present in the environment. More particularly, various embodiments relate to methods and systems of detecting a threatening or dangerous analyte that may increase survivability of an individual or group of individuals.

Aspects and embodiments may be directed to a system for detecting an analyte. The system may comprise an electrically insulating substrate including a trench formed therein, the trench having a first edge and a second edge. The system may also comprise a selective coating positioned on the electrically insulating substrate and at least one side of the trench, and constructed and arranged to selectively adsorb or absorb the analyte, wherein upon selectively adsorbing or absorbing the analyte, the selective coating expands. The system may also comprise an electrical conductor positioned on the selective coating having a first end proximate the first edge of the trench and a second end proximate the second end of the trench, wherein upon expansion of the selective coating, the first end of the electrical conductor contacts the second end of the electrical conductor.

The system may further comprise a radio frequency circuit connected to the electrical conductor. The system may also further comprise a power supply connected to the electrical conductor and the radio frequency circuit, wherein contact between the first end of the electrical conductor and the second end of the electrical conductor electrically connects the power supply to the radio frequency circuit. The analyte may comprise at least one of a chemical agent and a biological agent. In certain aspects, the analyte may be a chemical agent comprising a nitroaromatic compound. The nitroaromatic compound may comprise 2, 4, 6-trinitrotoluene. The selective coating may comprise poly(4-vinylpyridine). The electrically insulating substrate may be silicon. The electrical conductor may comprise a material selected from the group consisting of gold, palladium, silver, copper, aluminum, and combinations thereof.

Aspects and embodiments may be directed to a method of preparing a sensor for detecting an analyte. The method may comprise providing an electrically insulating substrate comprising a trench, the trench having a first end and a second end. The method may also comprise applying a selective coating to at least a portion of the electrically insulating substrate, the selective coating constructed and arranged to selectively adsorb or absorb the analyte, wherein upon selectively adsorbing or absorbing the analyte, the selective coating expands. The method may also comprise applying an electrically conductive material to at least a portion of the selective coating, the electrically conductive material having a first end proximate the first edge of the trench and a second end proximate the second end of the trench wherein upon expansion of the selective coating, the first end of the electrical conductor contacts the second end of the electrical conductor.

The method of preparing a sensor for detecting an analyte may comprise a method of preparing a sensor for detecting an analyte including at least one of a chemical agent and a biological agent. The method of preparing a sensor for detecting an analyte may comprise a method of preparing a sensor for detecting an analyte including a chemical agent comprising a nitroaromatic compound. The method of preparing a sensor for detecting an analyte may comprise a method of preparing a sensor for detecting an analyte comprising 2, 4, 6-trinitrotoluene.

Certain aspects may be directed to a method wherein applying a selective coating to at least a portion of the electrically insulating substrate may comprise applying a selective coating comprising poly(4-vinylpyridine). Applying the electrically conductive material to at least a portion of the selective coating may comprise applying an electrically conductive material comprising a material selected from the group consisting of gold, palladium, silver, copper, aluminum, and combinations thereof. Providing the electrically insulating substrate comprising a trench may comprise providing a silicon substrate.

The method may further comprise connecting a radio frequency circuit connected to the electrically conductive material. The method may further comprise connecting a power supply to the electrical conductive material and the radio frequency circuit, wherein contact between the first end of the electrical conductor and the second end of the electrical conductor electrically connects the power supply to the radio frequency circuit. The method may further comprise

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

Use of ordinal terms such as “first,” “second,” “third,” and the like in the specification and claims to modify an element does not by itself connote any priority, precedence, or order of one element over another or the temporal order in which acts of a method are performed, but are used merely as labels to distinguish one element having a certain name from another element having a same name, but for use of the ordinal term, to distinguish the elements.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1 is a functional block diagram of one example of a sensor system according to aspects of the invention;

FIG. 2A is an example of sensing element according to aspects of the invention;

FIG. 2B is a sensing element according to aspects of the invention;

FIG. 3A is an image of an example of the sensing element taken before analyte (NB) exposure according to aspects of the invention;

FIG. 3B is an image of an example of the sensing element taken before analyte exposure according to aspects of the invention;

FIG. 3C is an image of an example of the sensing element taken after analyte exposure according to aspects of the invention;

FIG. 3D is an image of an example of the sensing element taken after analyte exposure according to aspects of the invention;

FIG. 4 is a graph showing detection times for different concentrations of TNT according to aspects of the invention;

FIG. 5 is a functional block diagram of one example of an RF transmitter according to aspects of the invention;

FIG. 6 is a graph of range equation results for an example of a transmit element according to aspects of the invention; and

FIG. 7 is a graph of range equation results for an example of a transmit element according to aspects of the invention.

### DETAILED DESCRIPTION

A method and system is provided to detect an analyte present in the environment. The analyte may be a component, material, or chemical constituent of interest to an individual or entity. The system may be provided to detect the presence of a threatening or dangerous analyte and may have the ability to increase survivability of an individual or group of individuals due to detection of the threatening, or dangerous analyte. In some embodiments, a sensing device may be configured or constructed to detect an analyte that may be a chemical agent or a biological agent.

Explosive ordinance in the form of, for example, bombs, mines, and Improvised Explosive Devices (LEDs) pose a primary threat to military personnel, and may also be a threat to civilians. For example, over 120 million unexploded land mines are distributed across approximately 70 countries according to the International Committee for the Red Cross. Devices with an explosive typically contain a chemical agent. For example, nitroaromatic compounds such as 2, 4, 6-trinitrotoluene (TNT), are common in military grade and civil explosives, including LEDs. In certain examples, the sensing device is configured to detect chemical agents such as nitroaromatic compounds or other materials or substances that may be used in explosives.

Analytes comprising nitroaromatic compounds may be difficult to detect using vapor sensors. This may be due to their low vapor pressures relative to more easily detectable substances. The equilibrium concentration of TNT is 6.3 parts-per-billion (ppb) at 20° C., yet the concentration level in proximity to a landmine or an IED can be orders of magnitude lower due to encapsulation of the source or atmospheric dilution. In order to detect these systems or an approaching force carrying them, a device must have a minimum level of sensitivity to be effective. It may be beneficial to provide a device that may remain in a particular location for a predetermined period of time, for example, hours, days, weeks, months, or years. This device may allow for collection and accumulation of a particular analyte over the predetermined period of time, such that the concentration of the analyte that is detected may increase over the predetermined period of time. It may also be beneficial to provide a device that is not detectable, and thereby avoidable, by the individual or entity providing the threat, particularly where the device may remain in a particular location for an extended time period. For example, the device may be at least partially disguised or camouflaged to blend in with the environment it is placed.

Other analytes may include biological agents. For example, biological agents may include one or more bacterium, virus, prion, fungus, or toxin which may cause an infection, allergy, toxicity or otherwise create a hazard to human health. This may include agents such as anthrax (Bacillus anthracis), cholera (Vibrio cholerae), and Plague (Yersinia pestis).

In certain embodiments, a sensing element system is provided that can detect analytes of interest, such as those comprising nitroaromatic compounds or other chemical or biological agents, with detection capabilities as low as 0.95 parts per billion (ppb) and 3 femtograms (fg) in some examples. As discussed further below, the sensing element system may be integrated with transmitters and power supplies to produce a stand-alone sensor system which may be easily deployed and covert. According to one embodiment, the sensing element acts as a switch which may function to turn on the transmitter once detection is made, thus reducing or eliminating the need to power the transmitter prior to detection. The sensor system may be monitored remotely for periods of times in an automated manner, for example, the sensor system may be monitored for hours, days, weeks, months, or years.

One problem that is frequently encountered with conventional sensor systems is that the rate of power consumption is too high to be practically used for many applications. For example, as discussed above, to detect very low concentrations of analytes, it may be desirable to deploy sensor systems to monitor locations for extended periods of time, and therefore power consumption may be an important consideration.

Accordingly, in certain embodiments, there is provided a cost-effective stand-alone distributed sensor system that incorporates a method of eliminating battery drain until detection of a chemical or biological agent of interest occurs. As discussed further below, a sensing element is provided in which a chemically selective material (responsive to one or more analytes of interest) is used to transduce chemical reactions, namely detection of the analyte(s) of interest, into mechanical responses. In this manner, the sensing element may act as a “smart switch” that actuates in response to detection of the analyte and can be used to turn on a transmitter, as discussed above. Through the use of these smart switches, power drain in the sensor system may be driven to zero (or close to zero) until detection of a threat in the form of a chemical or biological agent occurs.

It is to be appreciated that embodiments of the methods and devices discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

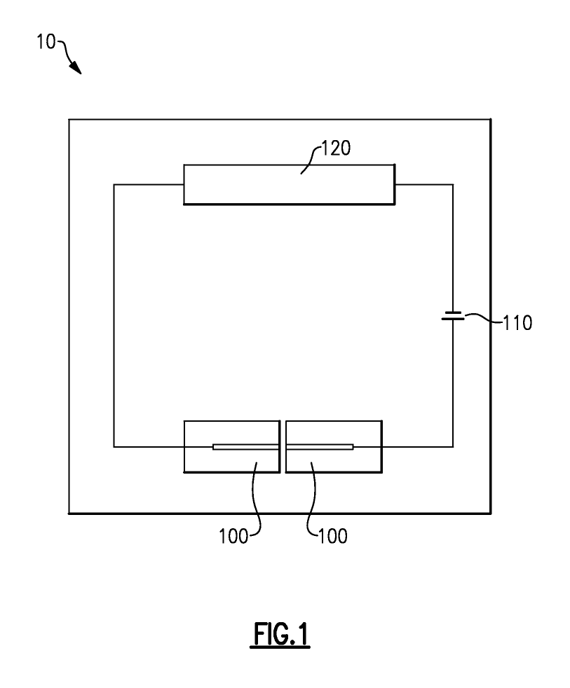

Referring to FIG. 1, there is illustrated a functional block diagram of one example of a sensor system according to certain embodiments. The sensor system 10 includes a sensing element 100, a power supply 110 (such as a battery, for example), and a transmitter 120, which may include radio frequency (RF) circuitry, for example, as discussed below. The sensing element 100 is coupled between the power supply 110 and the transmitter 120, and acts as a smart switch to close the circuit between the power supply and the transmitter, thereby providing power to and turning on the transmitter, responsive to detection of a specified analyte.

To reduce the possibility of false readings or alarms, the sensing element may be configured to achieve adequate selectivity such that it responds generally only to specific analytes. For example, the sensing element may include a chemically selective coating that may be sensitive or selective to only the analyte material or compound that the device is intended to detect. The chemically selective coating may be in the form of a material that may comprise a polymer material. The coating may be in the form of a polymer thin film. In some examples, the coating may be in the form of self-assembled monolayers. The coating may be, for example poly(4-vinylpyridine) (P4VP). Types of interactions that may occur in the coating may include charge transfer complexes and p-p stacking. The material that may be used may not undergo hydrogen bonding with nitroaromatics. For example, P4VP does not typically undergo hydrogen bonding with nitroaromatics. The chemical compound name for P4VP is C₂₁H₂₁N₃X₂, wherein X can be any element or compound that provides the desired properties. For example, X can be nitrogen.

As discussed above with regard to FIG. 1, the devices according to certain examples may comprise a power supply 110 that may be battery powered. The battery used may be a disc battery or a watch battery. The device may also use other types of batteries that may be smaller in size than disc or watch batteries. Other battery types that may be used include radio isotope batteries.

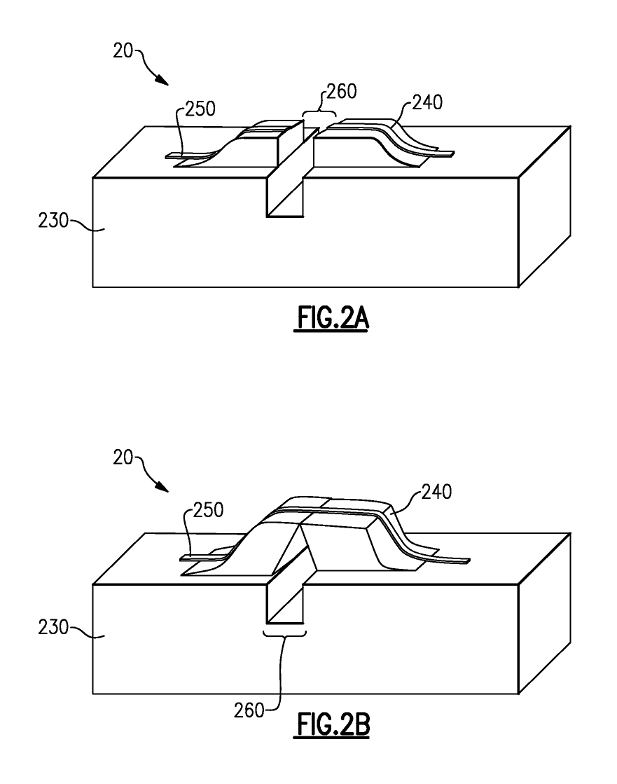

As shown in FIG. 2(a), in one example, the sensing element 100 comprises an electrically insulating substrate 230, a selective coating material 240, such as a chemically selective coating material, and an electrical conductor or electrically conductive layer 250. A trench 260 is provided in the electrically insulating substrate 230, such that the selective coating material 240 and the electrical conductor 250 are disposed on either side of the trench 260, but initially not in contact with one another, as shown in FIG. 2(a) and discussed further below. The trench 260 may be etched into the substrate 230. In certain embodiments, the substrate 230 may be a silicon substrate.

The selective coating 240 may be applied to the surface of the substrate 230. The selective coating may additionally be applied to the side walls of the trench (not shown in FIG. 2). The selective coating 240 may be applied by initiated chemical vapor deposition (iCVD), or any other suitable polymer coating application process. The electrical conductor 250 may be applied on top of a selective coating 240, as shown in FIG. 2(a). The electrical conductor 250 may be applied in a uniform manner, or may be applied nonconformally. The electrically conductive layer 250 may comprise any material suitable for allowing adequate conductance of electrical current. In certain examples, at least one of gold and palladium may be used. Other materials that may be used as the electrically conductive layer may include silver, copper, and aluminum. The electrically conductive layer may additionally be deposited onto the electrically insulating substrate. The electrically conductive layer may be deposited using sputter-coating techniques. The electrically conductive layer may be deposited such that initially an open circuit condition exists between the electrical conductors on either side of the trench. That is, the electrical conductor does not create a continuous layer inside or across the trench defined by the electrically insulating substrate.

In certain embodiments, the selective coating may swell or expand when the selective coating absorbs or adsorbs an analyte. For example, a chemical agent or biological agent related to a threat, may preferentially absorb onto or absorb into the coating. The expansion of the selective coating may allow the electrically conductive material on opposite sides of the trench to come into contact and create an electrical short. This is shown in FIG. 2(b), for example. As shown, the selective coating 240 of the sensing element 100 has expanded to allow the electrical conductor 250 on opposite sides of the trench 260 to come into contact and create an electrical short circuit. The creation of the electrical short may close the electrical circuit between the sensing element 100 and the transmitter 120, as shown in FIG. 1, and turn on the transmitter, as discussed above. The width of the trench 260 may be selected so as to provide for an adequate separation of the two sections of the selective coating 240 until the coating 240 comes into contact with the selected threat. In certain embodiments, the width of the trench may be in a range of about 400 nanometers (nm) to about 600 nm. In certain other embodiments, the width of the trench may be in the range of about 60 micrometers to 200 micrometers. Thus, the trench, in combination with the expandable chemically selective coating, may be used as a switch in which closure is due to detection of an analyte and allows the sensor system to begin to transmit a signal. In certain embodiments, the selective coating may be “re-shrunk” after expansion, for example, after a predetermined time period has elapsed, to disconnect the electrical circuit and allow for reuse of the sensor system.

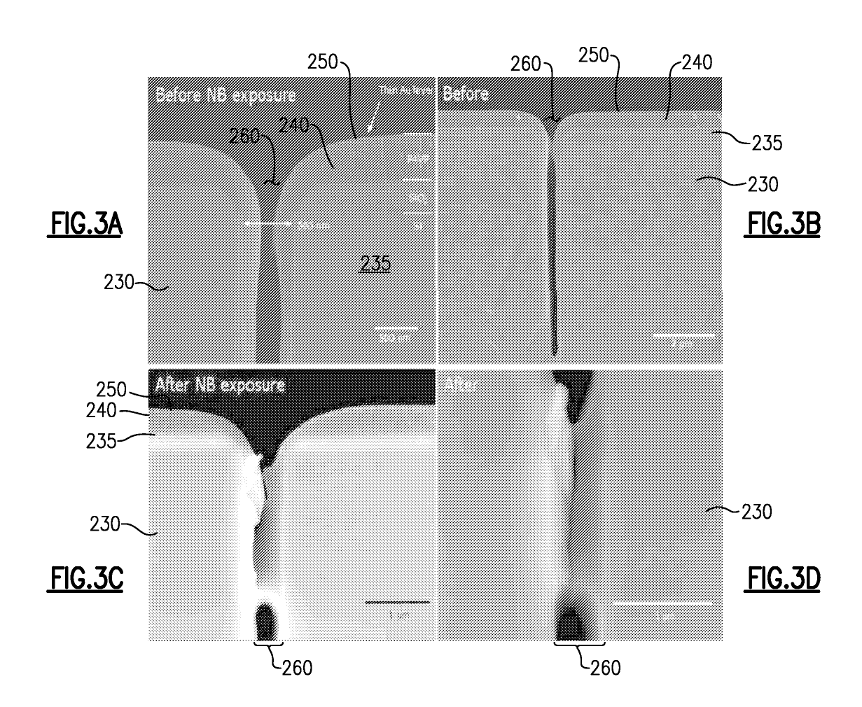

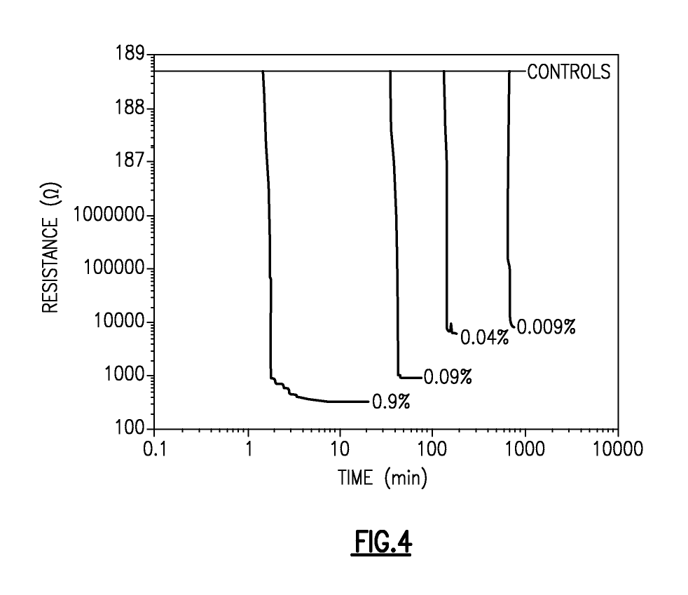

Referring to FIGS. 3A-3D, there are illustrated several images of an example of the sensing element taken before analyte (NB) exposure (FIGS. 3A and 3B) and after analyte exposure (FIGS. 3C and 3D) that demonstrate the expansion of the selective coating 240 after coming in contact with an analyte. In this example, NB is an explosive material comprising a nitroaromatic compound. In the example illustrated in, for example, FIG. 3A, the sensing element included a substrate 230 of silicon having a top layer 235 of silicon dioxide. The chemically sensitive or selective coating 240 included a layer of P4VP deposited over the silicon dioxide layer and along the sides of the trench 260. The electrical conductor 250 included a thin layer of gold. The trench 260 (excluding the chemically selective coating) was approximately 565 nm wide. As shown in the images of FIGS. 3A-3D, the exposure to an analyte causes the chemically selective coating 240 to expand, and closes the gap between the two sides of the trench 260, as shown in FIGS. 3C and 3D. This allows contact to occur between the two ends of the thin gold layer 250 located on the selective coating material 240. This contact effects closing of the switch and thereby may allow transmittal of a signal to notify an individual or entity that an analyte, for example a chemical or biological threat, has been detected.

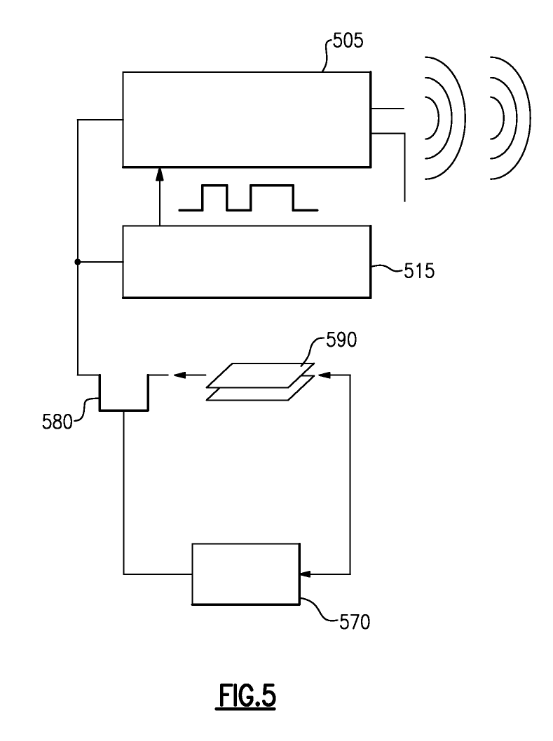

Referring to FIG. 4, there is illustrated a graph showing detection times for different concentrations of TNT. As shown in the plot of FIG. 4, greater concentrations of TNT may be detected in a shorter time period. In certain embodiments, the sensor system may be constructed and arranged to detect TNT at levels of about 1000 ppb. As discussed above, the sensors may be placed in an area, and they may remain in the same area for a period of time. Over this time period, they may collect or accumulate analyte. After a certain amount or concentration is collected or accumulated, the sensor may be activated and the switch may be turned on to send a signal to an RF transmitter.

In certain embodiments, the sensor system may include an indicator or indicating device which provides an alarm either directly on the sensor, or remotely to inform a user that a threat has been detected. The alarm may be an auditory or visual alarm, such as a light emitting alarm. Visual or audio alarms may be disadvantageous in circumstances where it is desired that the sensor system be covert. In one example a passive reflector may be used for the indicator, thus allowing the sensor to remain covert. However, a passive reflector must be interrogated to determine if detection has been made, and may therefore require line of sight access from the interrogator to the sensor. Similarly, visual indicators may also require an uninterrupted line of sight between the sensor and the remote user or control station.

Accordingly, it may be desirable to provide a sensor system that may be persistent and covert which may automatically transmit detection information when detection of the analyte has been made. Therefore, in certain embodiments, RF transmitters may be used to provide indication of detection of the analyte, as discussed above with reference to FIG. 1.

One example of an RF transmitter is shown in FIG. 5. RF transmitters may provide several advantages. For example, RF transmitters may be easily integrated with Complementary Metal Oxide Semiconductor (CMOS) electronics. The devices may be efficient at high microwave frequencies, and may transmit through many obstacles to provide accurate and timely notification of analyte exposure to the sensor. In addition, many RF transmitters, such as that illustrated in FIG. 5, are available as common “off the shelf” (COTS) components. In the example illustrated in FIG. 5, the RF transmitter includes a timer 570 coupled to a transistor 580 and an energy storage device 590, such as a capacitor, for example. The RF transmitter also includes an oscillator 505, for example, a CMOS oscillator that generates the RF carrier wave at a specified frequency, and optionally a modulator 515 that modulates information onto the RF carrier wave. A remote monitoring device may be configured to monitor for the specified RF frequency corresponding to one or more deployed sensors. In some examples, sensors deployed in different locations, or configured to detect different analytes, may be configured to transmit at slightly different RF frequencies, or have different data modulated onto the RF carrier wave, such that the remote monitoring station may identify which sensors (or which locations) have detected the analyte, and/or what analyte has been detected.

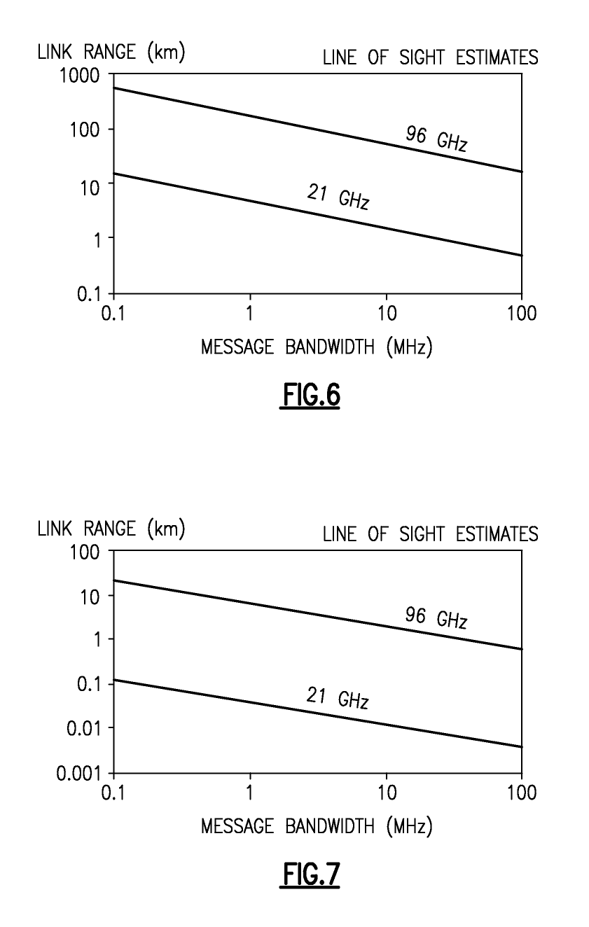

The ranges of the RF transmitters may be calculated based on their frequencies. The size of the antenna of the device may also impact its range. Sizes of the transmit element (antenna) may be any size suitable for the selected range. In certain embodiments, the size of the transmit element may be 0.2 inches by 0.06 inches. In another example, the transmit element size may be 0.03 inches by 0.03 inches. The range equation results for these example transmit elements are shown in FIGS. 6 and 7. FIG. 6 shows range equation results for a transmit element of 0.2 inches by 0.06 inches, at a 1 milliWatt peak output, a 1 Hz burst rate, and a 1 microsecond output time. FIG. 7 shows range equation results for a transmit element of 0.03 inches by 0.03 inches, at a 1 milliWatt peak output, a 1 Hz rate, and a 1 microsecond output.

Thus, aspects and embodiments may provide a small, cost-effective, stand-alone sensor system that may be deployed to covertly monitor for the presence of analytes of interest over time. As discussed above, the sensing element acts as a smart switch that automatically connects power to the indicator (for example, the RF transmitter) upon detection of the analyte, thus reducing or eliminating the need to power the indicator prior to detection, allowing for very long-life devices.

Having thus described various exemplary embodiments, many modifications, additions, and deletions may be made therein without departing from the spirit and scope of the disclosure and its equivalents. Those skilled in the art would readily appreciate, given the benefit of this disclosure, that the various parameters and configurations described herein are meant to be exemplary and that actual parameters and configurations will depend upon the specific application for which the systems and methods of the present disclosure are used. Those skilled in the art will recognize, or be able to ascertain using no more than routine experimentation, many equivalents to the specific embodiments described herein. For example, those skilled in the art may recognize that the device, and components thereof, discussed herein may further comprise a network of systems or be a component of a greater detection system. It is, therefore, to be understood that the foregoing embodiments are presented by way of example only and that, within the scope of the appended claims and equivalents thereto, the disclosed systems, devices and methods may be practiced otherwise than as specifically described. The present devices, systems, and methods are directed to each individual feature, device, system, or method described herein. In addition, any combination of two or more such features, systems or methods, if such features, systems or methods are not mutually inconsistent, is included within the scope of the present disclosure.

Further, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only. Further, the depictions in the drawings do not limit the disclosures to the particularly illustrated representations.

What is claimed is:

1. A system for detecting an analyte comprising:
an electrically insulating substrate including a trench formed therein, the trench having a first edge and a second edge;
a selective coating positioned on the electrically insulating substrate and at least one side of the trench, and constructed and arranged to selectively adsorb or absorb the analyte, wherein upon selectively adsorbing or absorbing the analyte, the selective coating expands; and
an electrical conductor positioned on the selective coating having a first end proximate the first edge of the trench and a second end proximate the second end of the trench, wherein upon expansion of the selective coating, the first end of the electrical conductor contacts the second end of the electrical conductor.

2. The system of claim 1, further comprising a radio frequency circuit connected to the electrical conductor.

3. The system of claim 2, further comprising a power supply connected to the electrical conductor and the radio frequency circuit, wherein contact between the first end of the electrical conductor and the second end of the electrical conductor electrically connects the power supply to the radio frequency circuit.

4. The system of claim 1, wherein the analyte comprises at least one of a chemical agent and a biological agent.

5. The system of claim 4, wherein the analyte is a chemical agent comprising a nitroaromatic compound.

6. The system of claim 5, wherein the nitroaromatic compound comprises 2, 4, 6-trinitrotoluene.

7. The system of claim 5, wherein the selective coating comprises poly(4-vinylpyridine).

8. The system of claim 1, wherein the electrically insulating substrate is silicon.

9. The system of claim 1, wherein the electrical conductor comprises a material selected from the group consisting of gold, palladium, silver, copper, aluminum, and combinations thereof.

10. A method of preparing a sensor for detecting an analyte comprising:
providing an electrically insulating substrate comprising a trench, the trench having a first end and a second end;
applying a selective coating to at least a portion of the electrically insulating substrate, the selective coating constructed and arranged to selectively adsorb or absorb the analyte, wherein upon selectively adsorbing or absorbing the analyte, the selective coating expands; and
applying an electrically conductive material to at least a portion of the selective coating, the electrically conductive material having a first end proximate the first edge of the trench and a second end proximate the second end of the trench wherein upon expansion of the selective coating, the first end of the electrical conductor contacts the second end of the electrical conductor.

11. The method of claim 10, wherein the method of preparing a sensor for detecting an analyte comprises a method of preparing a sensor for detecting an analyte including at least one of a chemical agent and a biological agent.

12. The method of claim 11, wherein the method of preparing a sensor for detecting an analyte comprises a method of preparing a sensor for detecting an analyte including a chemical agent comprising a nitroaromatic compound.

13. The method of claim 12, wherein the method of preparing a sensor for detecting an analyte comprises a method of preparing a sensor for detecting an analyte comprising 2, 4, 6-trinitrotoluene.

14. The method of claim 12, wherein applying a selective coating to at least a portion of the electrically insulating substrate comprises applying a selective coating comprising poly(4-vinylpyridine).

15. The method of claim 10, wherein applying the electrically conductive material to at least a portion of the selective coating comprises applying an electrically conductive material comprising a material selected from the group consisting of gold, palladium, silver, copper, aluminum, and combinations thereof.

16. The method of claim 10, wherein providing the electrically insulating substrate comprising a trench comprises providing a silicon substrate.

17. The method of claim 10, further comprising connecting a radio frequency circuit connected to the electrically conductive material.

18. The method of claim 17, further comprising connecting a power supply to the electrical conductive material and the radio frequency circuit, wherein contact between the first end of the electrical conductor and the second end of the electrical conductor electrically connects the power supply to the radio frequency circuit.

19. The method of claim 18, further comprising exposing the sensor to an environment for a predetermined period of time.
